(19) United States
(12) Patent Application Publication
(10) Pub. No.: US 2024/0325715 A1
(43) Pub. Date: Oct. 3, 2024

(54) MULTI-STAGE CONNECTOR DISLODGEMENT PREVENTION DEVICE

(71) Applicant: CareFusion 303, Inc., San Diego, CA (US)

(72) Inventors: Frank CAI, Ontario, CA (US); Jason Andrew WINE, Brea, CA (US)

(21) Appl. No.: 18/608,084

(22) Filed: Mar. 18, 2024

Related U.S. Application Data
(60) Provisional application No. 63/455,809, filed on Mar. 30, 2023.

(51) Int. Cl. A61M 39/10

(52) U.S. Cl. CPC A61M 39/1011; A61M 2039/1061

(57) ABSTRACT

A coupler including a first connector having a first outer piece with a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece. The first inner piece configured to axially move relative to the first outer piece to transition from a retracted position to a partially extended position. The first channel including a first occlusion hole forming a first fluid pathway through the first connector when the first inner piece is in the retracted position. In the extended position the first occlusion hole is blocked by the first seal resulting in the first fluid pathway being blocked.

### CROSS REFERENCE TO RELATED APPLICATION

This application claims the benefit of priority under 35 U.S.C. § 119 to U.S. Provisional Patent Application Ser. No. 63/455,809, entitled “MULTI-STAGE CONNECTOR DISLODGEMENT PREVENTION DEVICE”, filed on Mar. 30, 2023, the entire contents of which is incorporated herein by reference.

### FIELD OF THE INVENTION

The present disclosure generally relates to connectors, and, in particular, to connector couplings.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, tubing or catheters are coupled or secured to each other to allow fluid communication between various portions of tubing or catheters.

In some applications, such tubing or catheters may become dislodged due to improper securement and/or when the coupling is subject to forces greater than what the coupling is designed to withstand.

### SUMMARY

One or more embodiments of the present disclosure are directed to a coupler including a first connector having a first outer piece with a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position, the first channel including a first occlusion hole, and the first channel and the first occlusion hole forming a first fluid pathway through the first connector when the first inner piece is in the retracted position. In the extended position, the first occlusion hole is blocked by the first seal resulting in the first fluid pathway being blocked, the first inner piece transitioning to the partially extended position in response to a pullout force exceeding a first predetermined threshold.

In some embodiments, the coupler further includes a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position, the second channel including a second occlusion hole, and the second channel and the second occlusion hole forming a second fluid pathway through the second connector when the second inner piece is in the retracted position. In the partially extended position, the second occlusion hole is blocked by the second seal resulting in the second fluid pathway being blocked, the second inner piece transitioning to the partially extended position in response to the pullout force exceeding the first predetermined threshold.

In some embodiments, the coupler further includes a coupling element coupling the first connector to the second connector, the coupling element configured to cause the first fluid pathway to be in fluid communication with the second fluid pathway when the first connector is coupled to the second connector. The coupling element is configured to break into two or more portions in response to the pullout force exceeding a second predetermined threshold. The second predetermined threshold is greater than the first predetermined threshold.

In some embodiments, a central axis extends through the first connector, the coupling element, and the second connector when the first connector is coupled to the second connector via the coupling element.

In some embodiments, the first connector is configured to remain coupled to the coupling element and the second connector when the pullout force does not exceed the first predetermined threshold force.

In some embodiments, the coupler has a first configuration and in the first configuration the first connector is coupled to the second connector via the coupling element such that the first fluid pathway is in fluid communication with the second fluid pathway. The coupler also has a second configuration and in the second configuration the first connector is coupled to the second connector and the first inner piece is in the partially extended position and the second inner piece is in the partially extended position such that the first fluid pathway is blocked and the second fluid pathway is blocked. The coupler has a third configuration and in the third configuration the coupling element is broken such that the first connector is decoupled from the second connector.

In some embodiments, the first inner piece includes a first coupling portion and the first inner piece is configured to transition from the partially extended position to a fully extended position, the fully extended position being where the first coupling portion is disposed further from the first occlusion hole compared to when the first inner piece is in the partially extended position. The first inner piece includes one or more locking tabs, the one or more locking tabs configured to prevent retraction of the first inner piece when the first inner piece is in the fully extended position.

In some embodiments, the first inner piece transitions from the partially extended position to the fully extended position in response the pullout force exceeding a second predetermined threshold, the second predetermined threshold being greater than the first predetermined threshold.

In some embodiments, the first outer piece includes a first interior space and the first inner piece is biased to be in the retracted into the first interior space due to a vacuum generated in the first interior space.

In some embodiments, the first outer piece includes a first tubing portion configured to couple to a portion of tubing and the first inner piece includes a first coupling portion configured to couple to a coupling element, the first tubing portion being in fluid communication the first coupling portion through the first channel and the first occlusion hole when the first inner piece is in the retracted position.

In some embodiments, a length of the first inner piece is greater than a length of the first outer piece and a maximum diameter of the first outer piece is greater than a maximum diameter of the first inner piece.

In some embodiments, the coupler further includes a locking ring secured to the first outer piece, the locking ring configured to prevent the first inner piece from withdrawing from the first outer piece.

In some embodiments, the coupler further includes a biasing element disposed within an interior space of the first outer piece and having a compressed state and an expanded state, the biasing element configured to bias the first inner piece to be in the retracted position, wherein the biasing element transitions from the expanded state to the compressed state in response to the pullout force exceeding the first predetermined threshold.

In some embodiments, the pullout force is a force applied to the first connector along a central axis of the first connector and the central axis extends at least along a length of the first connector.

One or more embodiments of the present disclosure are directed to a coupler including a first connector having a first outer piece with a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position, the first channel including a first occlusion hole, and the first channel and the first occlusion hole forming a first fluid pathway through the first connector when the first inner piece is in the retracted position, wherein in the partially extended position the first occlusion hole is blocked by the first seal resulting in the first fluid pathway being blocked, the first inner piece transitioning to the partially extended position in response to a pullout force exceeding a first predetermined threshold, a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position, the second channel including a second occlusion hole, and the second channel and the second occlusion hole forming a second fluid pathway through the second connector when the second inner piece is in the retracted position. In the partially extended position, the second occlusion hole is blocked by the second seal resulting in the second fluid pathway being blocked, the second inner piece transitioning to the partially extended position in response to the pullout force exceeding the first predetermined threshold. The coupler further includes a coupling element coupling the first connector to the second connector, the coupling element configured to cause the first fluid pathway to be in fluid communication with the second fluid pathway when the first connector is coupled to the second connector.

One or more embodiments of the present disclosure are directed to a coupler including a first connector having a first outer piece with a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position to a fully extended position, the first channel including a first occlusion hole substantially perpendicular to the first channel, and the first channel and the first occlusion hole forming a first fluid pathway through the first connector when the first inner piece is in the retracted position, wherein in the partially extended position the first occlusion hole is blocked by the first seal resulting in the first fluid pathway being blocked, the first inner piece transitioning to the partially extended position in response to a pullout force exceeding a first predetermined threshold, wherein the first inner piece transitions from the partially extended position to the fully extended position in response the pullout force exceeding a second predetermined threshold, a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position to a fully extended position, the second channel including a second occlusion hole substantially perpendicular to the second channel, and the second channel and the second occlusion hole forming a second fluid pathway through the second connector when the second inner piece is in the retracted position, wherein in the partially extended position the second occlusion hole is blocked by the second seal resulting in the second fluid pathway being blocked, the second inner piece transitioning to the partially extended position in response to the pullout force exceeding the first predetermined threshold, wherein the first inner piece transitions from the partially extended position to the fully extended position in response the pullout force exceeding a second predetermined threshold, and a coupling element coupling the first connector to the second connector, the coupling element configured to cause the first fluid pathway to be in fluid communication with the second fluid pathway when the first connector is coupled to the second connector. The coupling element is configured to break into two or more portions in response to the pullout force exceeding the second predetermined threshold, the second predetermined threshold being greater than the first predetermined threshold. The pullout force is a force applied to the first connector along a central axis of the first connector and the central axis extends at least along a length of the first connector, the second connector, and the coupling element.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a system diagram showing a coupler assembly in use, in accordance with various aspects of the present disclosure.

FIG. 2A is a perspective side view of the coupler assembly of FIG. 1 in a first configuration, in accordance with various aspects of the present disclosure.

FIG. 2B is a perspective side view of the coupler assembly of FIG. 1 in a second configuration, in accordance with various aspects of the present disclosure.

FIG. 2C is a perspective side view of the coupler assembly of FIG. 1 in a third configuration, in accordance with various aspects of the present disclosure.

FIG. 3A is a perspective front view of a connector of the coupler assembly of FIG. 1 with the connector in the first configuration, in accordance with various aspects of the present disclosure.

FIG. 3B is a perspective front view of the connector of FIG. 3A with the connector in the second configuration, in accordance with various aspects of the present disclosure.

FIG. 4A is an exploded view of the connector of FIG. 3A, in accordance with various aspects of the present disclosure.

FIG. 4B is a cross-sectional view of the connector of FIG. 4A, in accordance with various aspects of the present disclosure.

FIG. 5A is a cross-sectional view of the connector of FIG. 3A with the connector in the first configuration, in accordance with various aspects of the present disclosure.

FIG. 5B is a cross-sectional view of the connector of FIG. 3A with the connector in the second configuration, in accordance with various aspects of the present disclosure.

FIG. 5C is a cross-sectional view of the connector of FIG. 3A with the connector in a third configuration, in accordance with various aspects of the present disclosure.

FIG. 6 is a graph of force and displacement of the coupler assembly of FIG. 1, in accordance with various aspects of the present disclosure.

FIG. 7A is a system diagram showing a coupler assembly of a second embodiment in use, in accordance with various aspects of the present disclosure.

FIG. 7B is a cross-sectional view of the connector of FIG. 7A with the connector in the first configuration, in accordance with various aspects of the present disclosure.

FIG. 7C is a perspective front view of the connector of FIG. 7A with the connector in the second configuration, in accordance with various aspects of the present disclosure.

FIG. 7D is a cross-sectional view of the connector of FIG. 7A with the connector in a third configuration, in accordance with various aspects of the present disclosure.

FIG. 8A is a cross-sectional view of a third embodiment of a connector of the coupler assembly of FIG. 1 with the connector in the first configuration, in accordance with various aspects of the present disclosure.

FIG. 8B is a cross-sectional view of a third embodiment of a connector of the coupler assembly of FIG. 1 with the connector in the second configuration, in accordance with various aspects of the present disclosure.

FIG. 9A is a cross-sectional view of a fourth embodiment of a connector of the coupler assembly of FIG. 1 with the connector in the first configuration, in accordance with various aspects of the present disclosure.

FIG. 9B is a cross-sectional view of a fourth embodiment of a connector of the coupler assembly of FIG. 1 with the connector in the second configuration, in accordance with various aspects of the present disclosure.

### DETAILED DESCRIPTION

The disclosed coupler assembly includes at least two connectors (e.g., a first connector and a second connector) and a coupling tube. The two connectors are identical to each other and the coupling tube connects one connector to the other. The connector is configured to couple to the other connector via the coupling tube. The coupler assembly may have a first configuration, a second configuration, and a third configuration. In the first configuration, the connector is coupled to the other connector via the coupling tube and a fluid pathway is formed between the connectors through the coupling tube to allow fluid to flow from the connector through the coupling tube to the other connector. In the second configuration, the connector is coupled to the other connector and the fluid pathway is blocked, preventing flow of fluid from the connector through the coupling tube to the other connector. In the third configuration, the fluid pathway remains blocked and the coupling tube is damaged, broken, or otherwise rendered unusable resulting in decoupling of the connector from the other connector.

The coupler assembly may be configured to couple a first portion of tubing to a second portion of tubing. For example, the first portion of tubing may be coupled to the first connector and the second portion of tubing may be coupled to the second connector. The first portion of tubing and/or the second portion of tubing may also couple to a patient or fluid source. In some embodiments, the coupler assembly allows for the flow of fluid from the first portion of tubing to the second portion of tubing. For example, the first connector may be coupled to the second connector such that a fluid pathway is formed through the first connector and the second connector via the coupling tube to allow the flow of fluid from the first portion of tubing through the first connector and the coupling tube to the second connector to the second portion of tubing.

In some embodiments, the first connector and second connector provide one way fluid flow. For example, when the first connector is coupled to the second connector via the coupling tube, fluid may flow from the first connector to the second connector and not from the second connector to the first connector. In some embodiments, decoupling of the first connector from the second connector (e.g., due to coupling tube breaking or rendered unusable) results in the flow of fluid from the first connector to the second connector ceasing, thereby preventing leakage when the first connector is decoupled from the second connector. In some embodiments, upon decoupling of the first connector from the second connector, the first connector is sterilized (e.g., via a sterilized cloth or a sterilizing device) or replaced with a new sterile connector to prevent infection or contamination that can occur if the first connector is re-used without sterilization. In some embodiments, upon breaking of coupling tube, which results in decoupling of the first connector from the second connector, the coupling tube is replaced and the first connector is recoupled to the second connector via the replaced coupling tube.

In some embodiments, the fluid pathway from the first connector through the coupling tube to the second connector is blocked in response to a pullout force being applied to the first connector or the second connector. When the pullout force exceeds a first predetermined threshold, the fluid pathway through the first connector and/or the second connector is blocked and the coupling tube remains intact. When the pullout force exceeds a second predetermined threshold, the fluid pathway remains blocked and the coupling tube breaks. In some embodiments, the second predetermined threshold is greater than the first predetermined threshold.

In some embodiments, the coupling tube breaks causing the first connector to decouple from the second connector based on a force that exceeds the second predetermined threshold force. When a force is applied to the first connector, such as the pullout force, that exceeds the second predetermined threshold force, the first connector may decouple from the second connector due to the coupling tube breaking. The pullout force may be a force that occurs along the longitudinal axis of the first connector, the second connector, and/or the coupling tube. In some embodiments, the pullout force is caused by tugging or pulling on the first portion of tubing coupled to the first connector. Alternatively, the pullout out force applied to the first connector may be caused by tugging or pulling on the second connector and/or the second portion of tubing coupled to the second connector.

In some embodiments, once the first connector is decoupled from the second connector, the first connector is configured to be re-coupled to the second connector. For example, once the first connector decouples from the second connector (e.g., due to a disconnection event), the first connector may be configured to allow for re-coupling to the second connector after a disconnection event due to replacing of the coupling tube.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the connection of medical fittings for the administration of medical fluid using the disclosed coupler, it is to be understood that this description is only an example of usage and does not limit the scope of the claims. Various aspects of the disclosed coupler may be used in any application where it is desirable to secure the connection of various tubing and fittings.

The disclosed coupler assembly overcomes several challenges discovered with respect to certain conventional couplers. One challenge with certain conventional couplers is that certain conventional couplers may be improperly secured. Further, during use, certain conventional couplers may be designed to release or dislodge in response to relatively low pullout forces. For example, certain conventional couplers may release in response to pullout forces experienced during patients rolling over in bed, patients catching tubing or lines on bed rails, moving patients to a different bed, fidgeting by pediatric patients, and/or disoriented adult patients pulling out their lines. Indeed, the Association for Vascular Access (AVA) Annual Scientific Meeting in 2017 reported a 10% dislodgement rate for 1,000 patients fitted with peripheral IV catheters, translating to approximately 33 million dislodgements per year in the U.S. alone. Because the accidental or unintentional dislodgement of tubing, catheters, or fittings may interrupt the administration of medical fluids, the use of certain conventional couplers is undesirable.

Therefore, in accordance with the present disclosure, it is advantageous to provide couplers and coupler/connector assemblies as described herein that allows for improved securement of fittings or connectors. The disclosed couplers and coupler/connector assemblies are structured as described herein so as to permit the secure retention of the first connectors, while allowing for decoupling after a disconnection event.

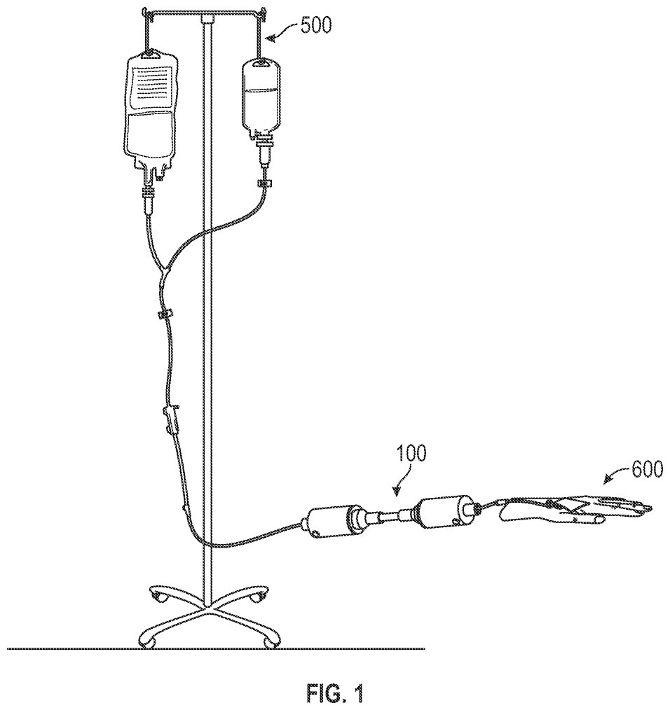

Referring to FIGS. 1-4B, first connector 102 may be structurally identical to second connector 102′. First connector 102 may be used interchangeably with second connector 102′ when coupler assembly 100 is described herein. Second connector 102′ may include all the elements of first connector 102. As used herein, elements of second connector 102′ that correspond with first connector 102 include “′”. For example, opening 110 refers to an opening of first connector 102 and opening 110′ refers to a corresponding identical opening of second connector 102′.

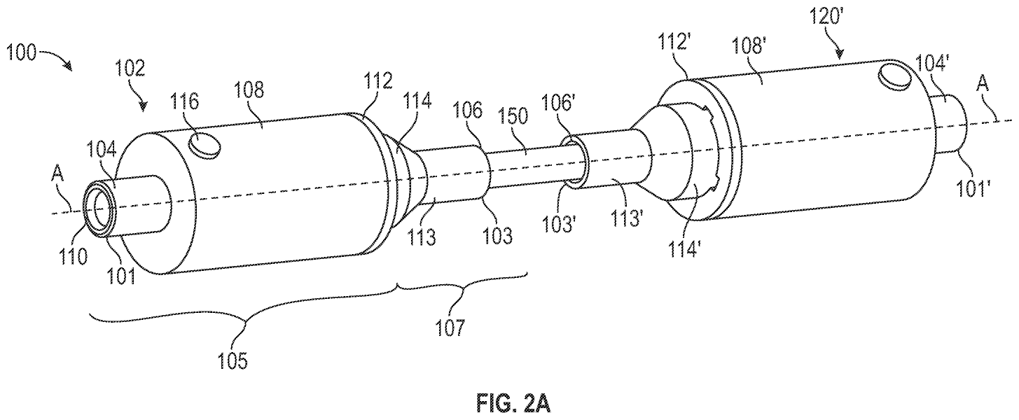

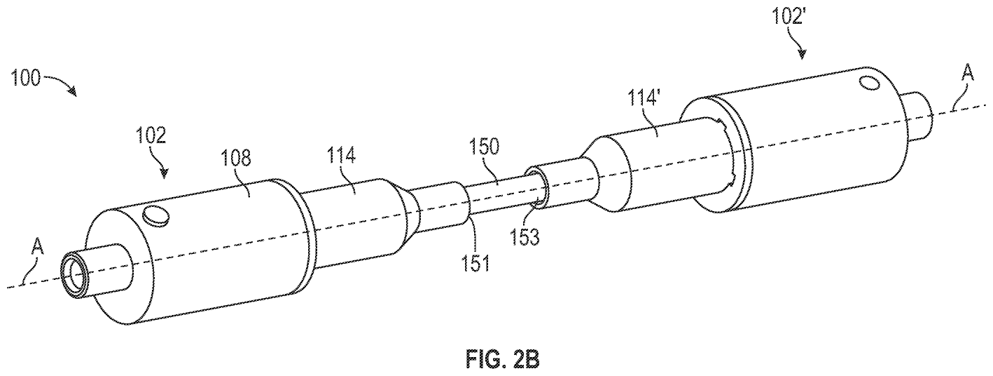

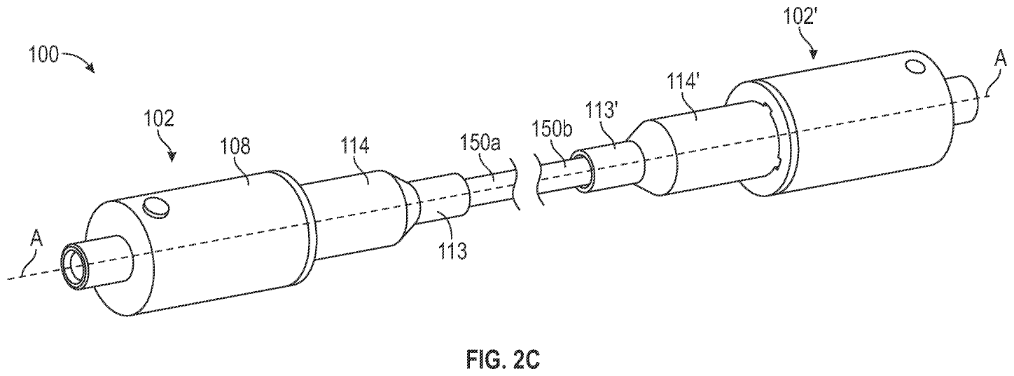

FIG. 1 is a system diagram showing a coupler assembly in use, in accordance with various aspects of the present disclosure. FIG. 2A is a perspective side view of the coupler assembly of FIG. 1 in a first configuration, in accordance with various aspects of the present disclosure. FIG. 2B is a perspective side view of the coupler assembly of FIG. 1 in a second configuration, in accordance with various aspects of the present disclosure. FIG. 2C is a perspective side view of the coupler assembly of FIG. 1 in a third configuration, in accordance with various aspects of the present disclosure.

With reference to FIGS. 1-2C, coupler assembly 100 allows the flow of a fluid, such as a medical fluid, from fluid source 500 to patient end 600 by releasably coupling a portion of tubing or line with another portion of tubing or line in fluid communication. Coupler assembly 100 may include first connector 102, coupling element 150, and second connector 102′. In some embodiments, second connector 102′ is identical to first connector 102.

First connector 102 may be configured to couple to second connector 102′ via coupling element 150. In some embodiments, first connector 102 and/or second connector 102′ are one way connectors. In the depicted example, portions of tubing can be terminated with connectors/valves, such as first connector 102 and/or second connector 102′. In some embodiments, fluid from fluid source 500 flows through coupler assembly 100 to patient end 600. A cannula or needle may be inserted within a patient at patient end 600 allowing medical fluid to flow from fluid source 500 through coupler assembly 100 and into a patient at patient end 600. In some embodiments, the flow of fluid from fluid source 500 to patient end 600 is interrupted or prevented in response to a force being applied to first connector 102 and/or second connector 102′. In some embodiments, decoupling of first connector 102 from second connector 102′ due to breaking of interrupts or prevents flow from fluid source 500 to patient end 600.

In some embodiments, coupler assembly 100 includes central axis A-A and first connector 102, coupling element 150, and second connector 102′ are coupled in series along central axis A-A. First connector 102, coupling element 150, and second connector 102′ may allow for the connection and/or disconnection of tubing to allow for selective fluid communication therebetween. Central axis A-A may extend longitudinally along the length of first connector 102, coupling element 150, and second connector 102′.

Coupler assembly 100 may have a first configuration (FIG. 2A), a second configuration (FIG. 2B), and a third configuration (FIG. 2C). In the first configuration, first connector 102 is coupled to second connector 102′ via coupling element 150 and a fluid pathway from first connector 102 to second connector 102′ through coupling element 150 is formed. In the second configuration, first connector 102 is coupled to second connector 102′ via coupling element 150 and a portion (e.g., inner piece 107) of first connector 102 and a portion (e.g., inner piece 107′) of second connector 102′ are each in an extended position causing the fluid pathway to be interrupted or blocked preventing fluid to flow from first connector 102 through coupling element 150 to second connector 102′. In some embodiments, coupler assembly 100 transitions from the first configuration to the second configuration in response to a pullout force exceeding a first predetermined threshold. The pullout force may be applied to first connector 102 and/or second connector 102′. In the third configuration, first connector 102 is decoupled to second connector 102′ due to coupling element 150 breaking in response to a pullout force exceeding a second predetermined threshold. In the third configuration, the portion of first connector 102 and the portion of second connector 102′ are each in the extended position and the fluid pathway remains interrupted or blocked preventing fluid to flow from first connector 102 to second connector 102′.

In some embodiments, coupler assembly 100 transitions from the first configuration to the second configuration in response to a pullout force applied to first connector 102 and/or second connector 102′. When the pullout force exceeds a first predetermined threshold, but not the second predetermined threshold, coupler assembly 100 is in the second configuration. Upon an increase in the pullout force, coupler assembly 100 transitions from the second configuration to the third configuration. For example, when the pullout force exceeds the second predetermined threshold, coupling element 150 may break resulting in first connector 102 being decoupled from second connector 102′. In some embodiments, when coupler assembly 100 transitions from the second configuration to the third configuration, the portion of first connector 102 and the portion of second connector 102′ remain in extended position and the fluid pathway remains blocked or interrupted.

Referring to FIGS. 2A-2C, coupler assembly 100 may transition from the first configuration to the second configuration and from the second configuration to the third configuration. In the first configuration (FIG. 2A), first connector 102 is coupled to coupling element 150, which is coupled to second connector 102′. In the first configuration, a fluid pathway is formed between first connector 102 and second connector 102′ via coupling element 150. When coupler assembly transitions from the first configuration to the second configuration (FIG. 2B) in response to a pullout force exceeding a first predetermined threshold, but being less than the second predetermined threshold, a portion of first connector 102 and a portion of second connector 102′ transitions from an initial position to an extended position. This results in the fluid pathway between first connector 102 and second connector 102′ being interrupted or blocked. Upon an increase in the pull out, or separation, force resulting in the pullout force exceeding the second predetermined threshold, coupling element 150 may break (e.g., into coupling element portion 150a and coupling element portion 150b) resulting in first connector 102 being decoupled from second connector 102′ and coupler assembly 100 transitioning to the third configuration. In the third configuration, the fluid pathway remains blocked due to the portion of first connector 102 and the portion of second connector 102′ being in the extended position thereby preventing fluid from leaking out of coupling element 150.

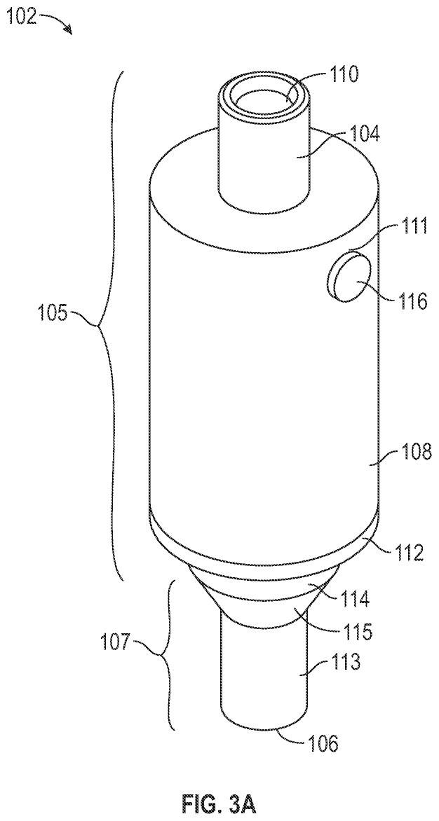

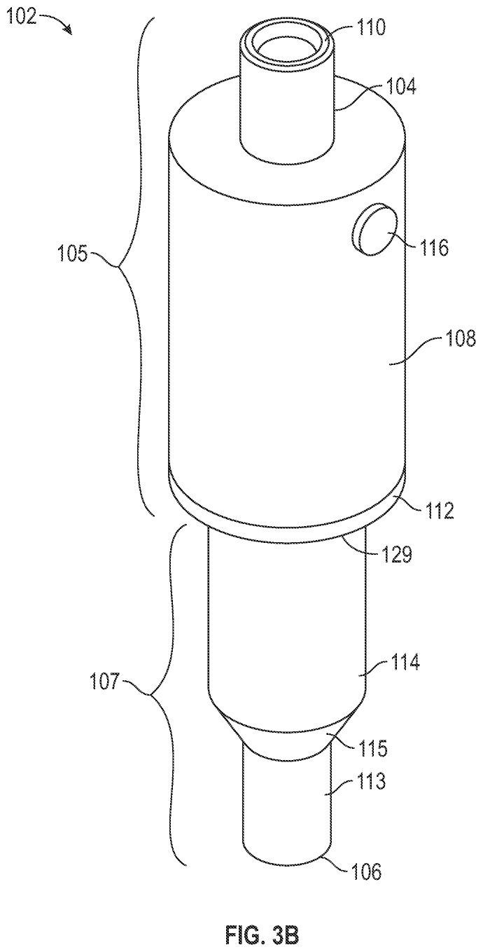

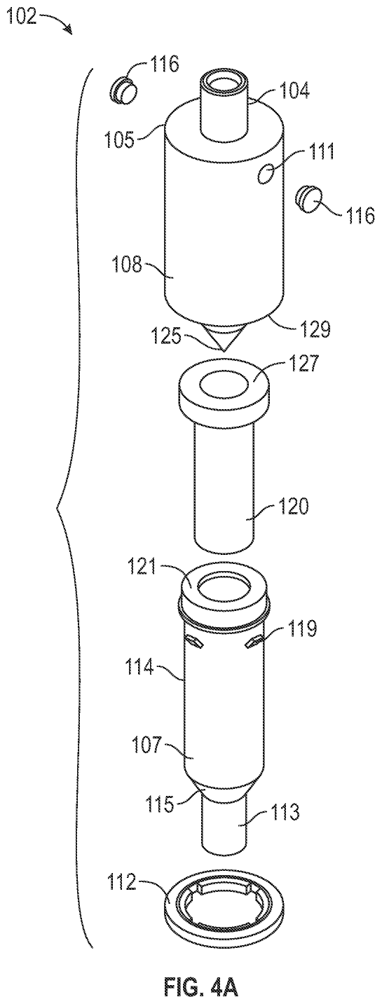

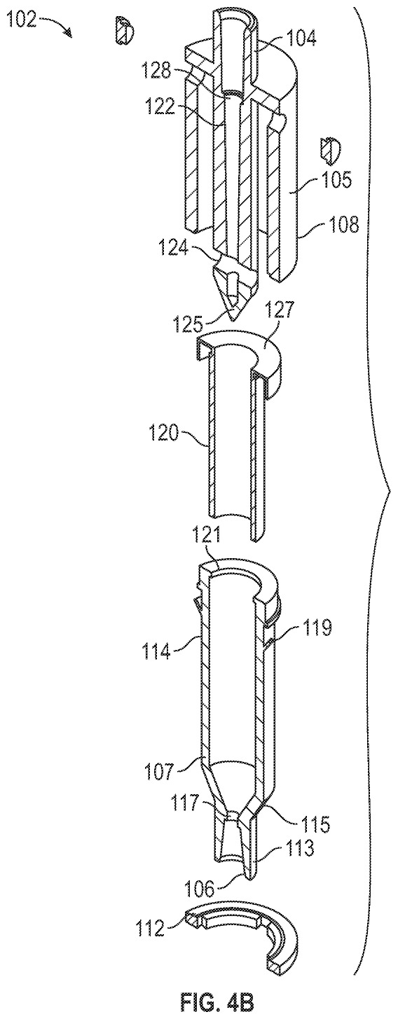

FIG. 3A is a perspective front view of a connector of the coupler assembly of FIG. 1 with the connector in the first configuration, in accordance with various aspects of the present disclosure. FIG. 3B is a perspective front view of the connector of FIG. 3A with the connector in the second configuration, in accordance with various aspects of the present disclosure. FIG. 4A is an exploded view of the connector of FIG. 3A, in accordance with various aspects of the present disclosure. FIG. 4B is a cross-sectional view of the connector of FIG. 4A, in accordance with various aspects of the present disclosure.

As discussed above, first connector 102 may be identical and used interchangeable with second connector 102′. Coupling element 150 may couple first connector 102 to second connector 102′. In some embodiments, first end 151 of coupling element 150 is coupled to second end 103 of first connector 102 and second end 153 of coupling element 150 is coupled to second end 103′ of second connector 102′ to couple first connector 102 to second connector 102′. Coupling element 150 may be substantially hollow to allow first connector 102 to be in fluid communication with second connector 102′. For example, fluid may flow from fluid source 500 through the fluid pathway of first connector 102 through coupling element 150 and through second connector 102′ to patient end 600.

In some embodiments, first connector 102 is coupled to a first portion of tubing to allow the first portion of tubing to be connected and/or disconnected with second connector 102′ via coupling element 150. First connector 102 may include first end 101 and second end 103. First end 101 may be coupled to tubing (e.g., a first portion of tubing) and second end 103 may be configured to couple to coupling element 150. For example, second end 103 of first connector 102 may be configured to couple to first end 151 of coupling element 150. In some embodiments, second end 153 of coupling element 150 is configured to couple to second end 103′ of second connector 102′. In some embodiments, a portion of tubing can be coupled with, or engage with first end 101 of first connector 102. First connector 102 via first end 101 may be in fluid communication with the tubing to allow fluid to pass through first connector 102.

In some embodiments, first end 101 can have a flat surface to allow for clinicians to easily clean and disinfect first end 101. First end 101 may be in fluid connection with second end 103. First end 101 and second end 103 may be disposed along the longitudinal length of first connector 102. For example, first end 101 and second end 103 may be disposed along central axis A-A. First end 101 and/or second end 103 may include an opening or channel to allow first end 101 and/or second end 103 to be in fluid communication with one or more elements (e.g., tubing, connectors, valves, collars, attachments, etc.). For example, first end 101 may be coupled to a tube and second end 103 may include opening 106 to allow for fluid communication through first connector 102 and fluid flow out of first connector 102. Opening 106 may be configured to be in fluid communication with coupling element 150, which is in fluid communication with opening 106′ of second connector 102′ to allow for fluid flow from first connector 102 to second connector 102′.

In some embodiments, first connector 102 is configured to couple to second connector 102′ via coupling element 150 such that fluid flows into first connector 102, through coupling element 150, and out of second connector 102′. For example, first connector 102 may include an opening (e.g., opening 110) configured to receive fluid and second connector 102′ may include an opening (e.g., opening 110′) configured to allow fluid to exit second connector 102′. In some embodiments, first connector 102 being coupled to second connector 102′ and coupler assembly 100 being in the first configuration results in the opening 110 of first connector 102 being in fluid communication with opening 110′ of second connector 102′.

In some embodiments, fluid can exit or flow through first connector 102 via second end 103 disposed opposite to first end 101. The flow path through first connector 102 can have a straight fluid pathway to make flushing easier and to reduce the risk of hemolysis. Optionally, first connector 102 can include features (e.g., raised features, gripping features) disposed on the outer surface of first connector 102 to allow a clinician to more easily handle or manipulate first connector 102. Some embodiments of first connector 102 may provide connectors that are compatible with connectors of other portions of fluid delivery systems. First connector 102 may be substantially cylindrically shaped.

In some embodiments, coupling element 150 is configured to break when coupler assembly 100 transitions to the third configuration (e.g., when inner piece 107 is in the fully extended position as described below). Coupling element 150 may be configured to break when the pullout force exceeds a second predetermined threshold force. In some embodiments, coupler assembly 100 has a greater overall length in the second configuration compared to when coupler assembly 100 is in the first configuration. When coupler assembly 100 is in the third configuration, coupling element 150 may break. For example, when the pullout force on first connector 102 and/or second connector 102′ exceeds the second predetermined threshold, coupling element 150 may break into two or more portions (e.g., coupling element portions 150a and 150b) resulting in coupler assembly 100 needing to be replaced. Coupling element 150 breaking prevents injury to the patient due to excessive tugging or pulling resulting in an increased pullout force on first connector 102 and/or second connector 102′. For example, if coupling element 150 did not break, the excessive tugging or pulling by the patient results in a greater pullout force (e.g., force exceeding the second predetermined threshold) being applied to coupler assembly 100. This may damage the tubing or other devices coupled to the patient, in addition to causing injury or pain to the patient due to coupler assembly 100 being coupled to the patient via a catheter or needle.

Referring to FIGS. 3A-3B, first connector 102 includes outer piece 105 and inner piece 107. Outer piece 105 may be configured to receive at least a portion of inner piece 107. In some embodiments, inner piece 107 has an initial position or retracted position (FIG. 3A), a partially extended position, and a fully extended position (FIG. 3B). In the initial position, a majority of inner piece 107 is disposed within outer piece 105 and inner piece 107 is unable to axially move towards first end 101. Inner piece 107 may be in the partially extended position when inner piece 107 is no longer in the initial position, but prior to reaching the fully extended position. As described in detail below, when inner piece 107 is in the partially extended position and/or the fully extended position, the overall length of first connector 102 increases and second end 103 is further away from first end 101 compared to when inner piece 107 is in the initial position. In other words, the distance between opening 110 and opening 106 is increased when inner piece 107 is in the partially or fully extended position compared to when inner piece 107 is in the initial position. Inner piece 107 may transition from the initial position to the partially extended position to the fully extended position in response to a pullout force. In some embodiments, inner piece 107 transitions from the initial position to the partially extended position when the pullout force is greater than the first predetermined threshold and transitions from the partially extended position to the fully extended position when the pullout force is greater than the second predetermined threshold.

In some embodiments, first connector 102 has an overall length of approximately 2 inches when inner piece 107 is in the initial position. First connector 102 may have an overall length of 0.5 inches to 6 inches, 1 inch to 4 inches, or 1.5 inches to 3 inches when inner piece 107 is in the initial position. In some embodiments, inner piece 107 is configured to extend greater than 0.5 inches. For example, the length of first connector 102 may increase greater than 0.5 inches when inner piece 107 transitions from the initial position to the fully extended position. First connector 102 may increase 0.1 inches to 5 inches, 0.5 to 3 inches, or 1 inch to 2.5 inches.

In some embodiments, outer piece 105 includes tubing portion 104 and body 108. Tubing portion 104 may be disposed proximate first end 101 and may include opening 110. Tubing portion 104 may be configured to couple to a portion of tubing, such as a portion of tubing coupled to fluid source 500. Tubing portion 104 may be substantially cylindrical. In some embodiments, tubing portion 104 has a constant maximum diameter. In some embodiments, tubing portion 104 is configured to couple to fluid source 500 via a portion of first tubing and tubing portion 104′ is configured to couple to patient end 600 via a portion of second tubing. Tubing portion 104 may be in fluid communication with extending portion 114.

Body 108 may be coupled to tubing portion 104. In some embodiments, body 108 and tubing portion 104 for a unitary structure. Alternatively, body 108 is fixedly or removably coupled to body 108. Body 108 may be disposed proximate second end 103 compared to tubing portion 104. Body 108 may include apertures 111 configured to receive valves or plugs 116. In some embodiments, apertures 111 are configured to allow access to the interior of outer piece 105 to create a vacuum within interior space 123 of outer piece 105. Plugs 116 may be configured to seal apertures 111 once a vacuum is created within interior space 123. The vacuum created within interior space 123 of outer piece 105 may result in inner piece 107 being biased to be in the initial position. For example, due to the vacuum within outer piece 105 and inner piece 107 being at least partially disposed within outer piece 105, inner piece 107 may be biased to be in the initial position due to the vacuum. Inner piece 107 may transition to the partially extended position or the fully extended position and be extended away from outer piece 105 in response to a pullout force exceeding the first predetermined threshold. In some embodiments, the first predetermined threshold is a force that is greater than opposing force created by the vacuum. In some embodiments, body 108 includes opening 129 configured to receive inner piece 107. For example, inner piece 107 may be configured to extend out of and retract into opening 129.

First connector 102 may include inner piece 107. Inner piece 107 may be configured to go into and extend out of outer piece 105 via opening 129. Inner piece 107 may have an initial position, a partially extended position and a fully extended position. Inner piece 107 may be biased to be in the initial position due to the vacuum created within outer piece 105. In some embodiments, inner piece 107 includes second end 103. Second end 103 may be proximate to first end 101 when inner piece 107 is in the initial position compared to when inner piece 107 is in the partially or fully extended position.

Inner piece 107 may include extending portion 114 and coupling portion 113. Coupling portion 113 may be coupled to extending portion 114. In some embodiments, extending portion 114 and coupling portion 113 form a unitary structure. Extending portion 114 may be configured to axially move along central axis A-A into and out of outer piece 105. For example, a majority of extending portion 114 may be disposed within outer piece 105 when inner piece 107 is in the initial position.

Coupling portion 113 may be disposed proximate second end 103 and may include opening 106. Coupling portion 113 may include channel 117, which may be in fluid communication with opening 106. Opening 106 may be in fluid communication with opening 110. Coupling portion 113 may have a constant maximum diameter. In some embodiments, the maximum diameter of coupling portion 113 is substantially the same as the maximum diameter of tubing portion 104. In some embodiments, inner piece 107 includes tapered portion 115 disposed between extending portion 114 and coupling portion 113. Tapered portion 115 may decrease in maximum diameter from an area proximate extending portion 114 to an area proximate coupling portion 113. In some embodiments, a majority of extending portion 114 has a constant maximum diameter. The tapered portion of extending portion 114 may have a decreasing diameter as it approaches coupling portion 113. In some embodiments, the tapered portion tapers from the maximum diameter of extending portion 114 to the maximum diameter of coupling portion 113.

In some embodiments, outer piece 105 includes locking ring 112. Locking ring 112 may be configured to secure inner piece 107 within outer piece 105 (e.g., body 108). For example, locking ring 112 may prevent inner piece 107 from being removed from outer piece 105 and/or extending completely out of outer piece 105. In some embodiments, locking ring 112 is fixedly coupled to body 108. For example, locking ring 112 may be coupled to body 108 via ultrasonic welding, adhesives, magnets, fasteners, or other coupling elements. In some embodiments, when inner piece 107 is in the fully extended position, a portion (e.g., a top edge) proximate opening 121 of inner piece 107 abuts locking ring 112 thereby preventing inner piece 107 from extending out of outer piece 105 any further.

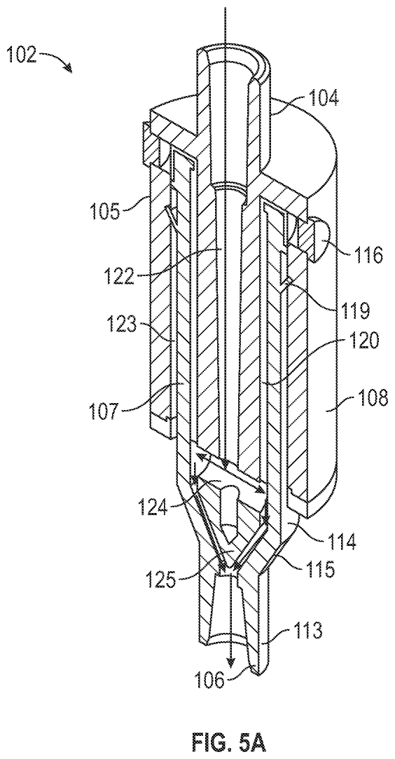

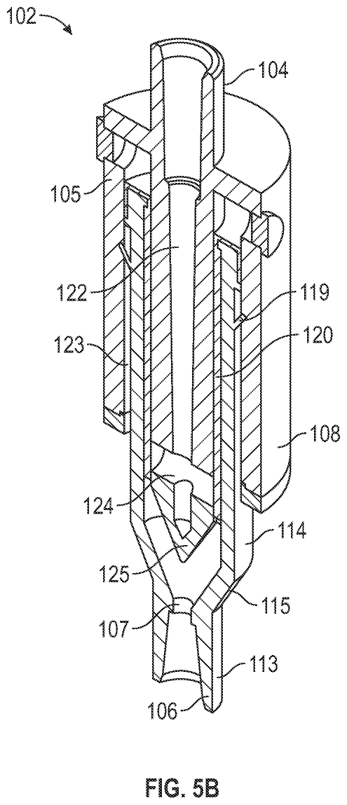

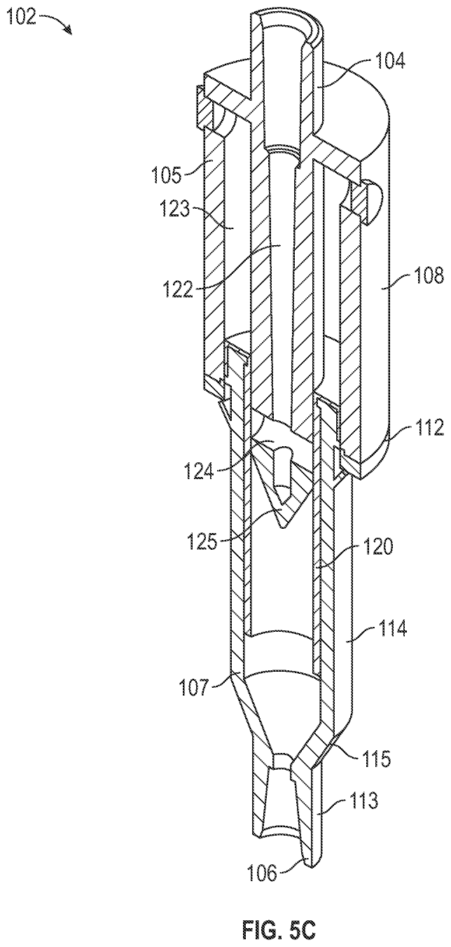

FIG. 5A is a cross-sectional view of the connector of FIG. 3A with the connector in the first configuration, in accordance with various aspects of the present disclosure. FIG. 5B is a cross-sectional view of the connector of FIG. 3A with the connector in the second configuration, in accordance with various aspects of the present disclosure. FIG. 5C is a cross-sectional view of the connector of FIG. 3A with the connector in a third configuration, in accordance with various aspects of the present disclosure.

Referring to FIGS. 4A-5C, coupler assembly 100 may include seal 120. Seal 120 may include top portion 127. Top portion 127 may be proximate first end 101 compared to the rest of seal 120. In some embodiments, seal 120 is received by opening 121 and seal 120 is disposed within inner piece 107. A portion of seal 120 proximate top portion 127 may receive a portion of inner piece 107 proximate opening 121 such a that a portion of inner piece 107 is disposed within a portion of top portion 127. In some embodiments, top portion 127 abuts and/or is proximate to opening 121. Seal 120 may be a rubber seal disposed within outer piece 105 and configured to be disposed within inner piece 107. In some embodiments, seal 120 is configured to move axially relative to outer piece 105. For example, seal 120 may be disposed within outer piece 105 and may be received by opening 121 of inner piece 107. In other words, seal 120 may be disposed within inner piece 107 and inner piece 107 may be disposed within outer piece 105. Opening 121 may be disposed opposite opening 106. In some embodiments, inner piece 107 extends from opening 121 to opening 106. Seal 120 may be configured to extend into and out of outer piece 105 with inner piece 107. For example, seal 120 and inner piece 107 may axially move relative to outer piece 105. In some embodiments, due to seal 120 being disposed within inner piece 107, movement of inner piece (e.g., axial movement) results in movement of seal 120.

In some embodiments, outer piece 105 includes channel 122 and interior space 123. Interior space 123 may be the space between channel 122 and the interior surface of outer piece 105. Channel 122 may be in fluid communication with opening 110. For example, fluid entering opening 110 via a portion of tubing coupled to first connector 102 (e.g., at tubing portion 104) may flow into opening and through channel 122. Channel 122 may have proximal end 128 and distal end 125. Proximal end 128 may be proximate first end 101 compared to distal end 125. Distal end 125 may include pointed geometry to allow for smoother flow of fluid out of channel 122. For example, distal end 125 may be substantially triangular shape to allow for smoother flow of fluid out of distal end 125.

Inner piece 107 may including one or more locking tabs 119. Locking tabs 119 may be disposed on an exterior of inner piece 107. For example, locking tabs 119 may extend from extending portion 114. In some embodiments, locking tabs 119 extend upward towards opening 121. Locking tabs 119 may be configured to prevent axially movement of inner piece 107 towards first end 101 when inner piece 107 is in the fully extended position. For example, when inner piece 107 is in the fully extended position, locking tabs 119 may be disposed outside of outer piece 105. When inner piece 107 is in the initial position or the partially extended position, locking tabs 119 may be disposed within outer piece 105 allowing inner piece 107 to axially move relative to outer piece 105. Once inner piece 107 transitions to the fully extended position, locking tabs 119 are no longer disposed within outer piece 105. When inner piece 107 is in the fully extended position, locking tabs 119 may about locking ring 112 preventing axially movement of inner piece 107 towards first end 101 of first connector 102. In some embodiments, locking tabs 119 are configured to be compressed to allow inner piece 107 to retract into outer piece 105 to transition inner piece 107 from the fully extended position to the initial position.

In some embodiments, inner piece 107 is configured to extend into and out of interior space 123. For example, when inner piece 107 is in the initial position, a majority of inner piece 107 is disposed within interior space 123. In some embodiments, seal 120 is disposed within inner piece 107 such that at least a majority of seal 120 and inner piece 107 are disposed within interior space 123 since seal 120 is at least partially disposed within inner piece 107. Inner piece 107 and seal 120 are configured to axially move within interior space 123 relative to outer piece 105. In some embodiments, when inner piece 107 is in the initial position, channel 122 is disposed within seal 120, which is disposed within inner piece 107, which is disposed within body 108 of outer piece 105 as shown in FIG. 5A. Seal 120 and inner piece 107 are configured to axially move relative to body 108, channel 122, and interior space 123.

Channel 122 may include occlusion hole 124. Occlusion hole 124 may be disposed proximate distal end 125. In some embodiments, occlusion hole 124 is transverse to channel 122. For example, channel 122 may be longitudinally disposed within outer piece 105 and occlusion hole 124 may be substantially perpendicular to channel 122. Occlusion hole 124 may allow fluid to flow out of channel. In some embodiments, when inner piece 107 is in the initial position, distal end 125 and occlusion hole 124 are disposed within tapered portion 115. Inner piece 107 may be biased to be in the initial position due to the vacuum created within interior space 123. For example, as illustrated in FIG. 5A, when inner piece 107 is in the initial position, fluid may flow through channel 122 and out of occlusion hole 124 through channel 117 of coupling portion 113 and out of opening 106. Occlusion hole 124 may remain unblocked to allow flow of fluid out of occlusion hole 124 through channel 122. Channel 122 may be configured to guide fluid flowing from occlusion hole 124 through opening 106.

Inner piece 107 may transition from the initial position (FIG. 5A) to the partially extended position (FIG. 5B) in response to a pullout force applied to first connector 102 and/or second connector 102′. In some embodiments, inner piece 107 transitions from the initial position to the partially extended position in response the pullout force exceeding a first predetermined threshold, but being less than the second predetermined threshold. Upon removal of the pullout force, inner piece 107 (and seal 120) transitions back to the initial position due the vacuum within interior space 123.

In the partially extended position, as illustrated in FIG. 5B, seal 120 and inner piece 107 may axially move relative to outer piece 105 such that top portion 127 of seal 120 and opening 121 of inner piece 107 move away from first end 101 toward distal end 125 of channel 122. In the partially extended position, top portion 127 and opening 121 are disposed between first end 101 and distal end 125 of outer piece 105.

In the partially extended position (FIG. 5B), seal 120 and inner piece 107 axially move away from first end 101 and towards distal end 125 of channel 122. Axially movement of seal 120 and inner piece 107 towards distal end 125 results in occlusion hole 124 being disposed within seal 120. For example, as inner piece 107 extends out of outer piece 105, seal 120 moves towards distal end 125 such that seal 120 blocks or occludes occlusion hole 124. Blockage or occlusion of occlusion hole 124 prevents the flow of fluid out of channel 122 thereby preventing flow of fluid out of first connector 102. In other words, blockage or occlusion of occlusion hole 124 interrupts or blocks the fluid pathway through first connector 102.

The blockage of occlusion hole 124 may interrupt the flow of fluid through first connector 102 causing an alarm to be generated by a device administer or controlling the flow of fluid. For example, fluid source 500 may be coupled to a device (e.g., a pump) to control the flow of fluid from fluid source 500 through coupler assembly 100 to patient end 600. Blockage or interruption of the fluid pathway through coupler assembly 100 may cause the device to administer an alarm such that a medical professional may assist the patient in unblocking the fluid pathway. For example, inner piece 107 may transition from the initial position, where a fluid pathway is formed and fluid flows from fluid source 500 through coupler assembly 100 to patient end 600, to the partially extended position in response to a pullout force exceeding the first determined threshold, but not exceeding the second predetermined threshold. The pullout force may be caused by the patient pulling or tugging on a tube coupled to first connector 102 and/or second connector 102′. When inner piece 107 is in the partially extended position due to a pullout force exceeding the first predetermined threshold but not the second predetermined threshold and occlusion hole 124 is blocked, the fluid pathway is blocked.

Inner piece 107 may transition from the partially extended position (FIG. 5B) to the initial position (FIG. 5A) in response the pullout force decreasing to below the first predetermined threshold and/or the pullout force ceasing. For example, a patient may tug or pull on tubing coupled to first connector 102 and/or second connector 102′. The pullout force caused by the tugging or pulling may be greater than the first predetermined threshold, but less than the second predetermined threshold. The tugging or pulling may cause inner piece 107 to transition from the initial position to the partially extended position thereby causing occlusion hole 124 to be blocked and the fluid pathway being blocked. A medical professional may assist the patient to stop the tugging or pulling thus decreasing or eliminating the pullout force resulting in inner piece 107 transition from the partially extended position (FIG. 5B) to the initial position (FIG. 5A) due to the vacuum created within interior space 123.

Inner piece 107 may transition from the partially extended position (FIG. 5B) to the full extended position (FIG. 5C) in response to the pullout force meeting or exceeding the second predetermined threshold force. In the fully extended position, opening 121 is disposed proximate locking ring 112 and occlusion hole 124. Once inner piece 107 is in the fully extended position, inner piece 107 may be prevented from axially moving relative to outer piece 105 to return to the partially extended position or the initial position due to locking tabs 119. In the fully extended position, locking tabs 119 prevent axial movement of inner piece 107 towards first end 101 and locking ring 112 prevents axial movement of inner piece 107 further away from first end 101.

In the initial position or the partially extended position, locking tabs 119 may be compressed radially inward. Once inner piece 107 is in fully extended position, locking tabs 119 may no longer be compressed and may abut locking ring 112 to prevent axial movement of inner piece 107 towards first end 101.

In some embodiments, when the pullout force meets the second predetermined threshold, inner piece 107 is fully extended and remain in the fully extended position due to locking tabs 119. The fluid pathway through first connector 102 remains blocked due to occlusion hole 124 being blocked by seal 120. In some embodiments, the pullout force exceeding the second predetermined threshold results in coupling element 150 breaking. For example, since inner piece 107 is in the fully extended position when the pullout force meets the second predetermined threshold, any increase in the pullout force (e.g., force exceeding the second predetermined threshold) results in coupling element 150 breaking. In some embodiments, when coupling element 150 breaks, a portion of coupling element 150 (e.g., portion 150a) remains with first connector 102 and a portion of coupling element 150 (e.g., portion 150b) remains with second connector 102′. When coupling element 150 breaks, there may not be any leakage of fluid due to the fluid pathway within first connectors 102 and/or second connector 102′ being blocked prior to coupling element 150 breaking.

In some embodiments, coupling element 150 is configured to deform when the pullout force exceeds the second predetermined threshold. For example, coupling element 150 may deform as the pullout force increasingly exceeds the second predetermined threshold. Coupling element 150 may continue to deform and then break as the pullout force further increases beyond the second predetermined threshold.

In some embodiments, inner piece 107 being in the initial position results in coupler assembly 100 being in the first configuration. Further, inner piece 107 being in the partially extended position results in coupler assembly 100 being in the second configuration. Inner piece 107 being in the fully extended position and coupling element 150 breaking results in coupler assembly 100 being in the third configuration. In some embodiments, coupler assembly 100 is configured to transition from the second configuration (e.g., inner piece 107 being in the partially extended position) back to the first configuration (e.g., inner piece 107 being in the initial position). For example, the pullout force may be decreased to under the first predetermined threshold or removed to allow inner piece 107 to retract into outer piece and transition from the partially extended position to the initial position resulting in coupler assembly 100 transitioning from the second configuration to the first configuration. However, coupler assembly 100 may be prevented from transitioning from the third configuration (e.g., inner piece 107 being in the fully extended position) to the second configuration (e.g., inner piece 107 being in the partially extended position). For example, locking tabs 119 and coupling element 150 breaking prevent coupler assembly from transitioning from the third configuration to the second configuration.

In some embodiments, once coupler assembly 100 is in the third configuration, a medical profession may replace coupler assembly 100 with a new coupler assembly 100. The new coupler assembly 100 may be in the first configuration such that a fluid pathway between fluid source 500 and patient end 600 is restored. In some embodiments, once coupler assembly 100 is in the third configuration, a user or medical professional compresses locking tabs 119 to cause inner piece 107 to retract into outer piece 105 to return to the initial position. The user or medical professional may then replace the broken coupling element 150 with a new coupling element 150 and recouple first connector 102 to second connector 102′ with the new coupling element 150 such that coupler assembly 100 returns to the first configuration.

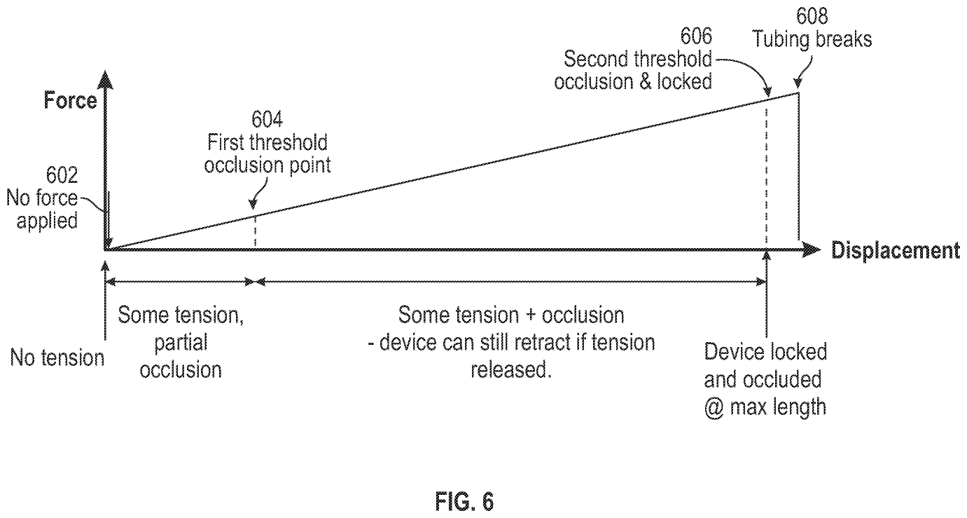

FIG. 6 is a graph of force and displacement of the coupler assembly of FIG. 1, in accordance with various aspects of the present disclosure.

Referring to FIG. 6, the relationship between the displacement of first connector 102 or second connector 102′ is provided. At point 602, there is no force (e.g., pullout force) applied to first connector 102, thus there is no displacement (e.g., increase in length) of first connector 102 and the fluid pathway formed in first connector 102 from opening 110 to opening 106 is open allowing for fluid flow through first connector 102. The vacuum within outer piece 105 keeps inner piece 107 from extending away from first end 101 when no force is applied.

As the force increases to point 604, there is some displacement of first connector 102 as inner piece 107 approaches the partially extended position, however the vacuum within outer piece 105 may prevent inner piece 107 from extending further such that seal 120 and inner piece 107 completely block occlusion hole 124. As the force approaches point 604, there may be partial occlusion or blockage of occlusion hole 124, however, the fluid pathway within first connector 102 is still open and allows for flow of fluid out of opening 106. At point 604, (e.g., the first predetermined threshold), inner piece 107 is in the partially extended position and occlusion hole 124 is blocked. When the force (e.g., pullout force) applied to first connector 102 reaches or exceeds point 604 (e.g., the first predetermined threshold) but prior to reaching point 606 (e.g., the second predetermined threshold), occlusion hole 124 is fully blocked by seal 120 and inner piece 107 resulting in the fluid pathway within first connector 102 being blocked.

As the force exceeds point 604, but prior to reaching point 606, inner piece 107 may still be configured to retract back into outer piece 105 due to locking tabs 119 not being engaged to abut locking ring 112. At point 606 (e.g., the second predetermined threshold), the fluid pathway within first connector 102 is blocked and inner piece 107 is in the fully extended position. At point 606, locking tabs 119 are engaged to prevent inner piece 107 from retracting into outer piece 105. Further, at point 606, first connector 102 is at its greater overall length.

As the force exceeds point 606, inner piece 107 is not able to extend further resulting in coupling element 150 becoming stretched and displaced. At point 608, where the force exceeds the second predetermined threshold, coupling element 150 breaks and coupler assembly 100 is required to be replaced. In some embodiments, coupling element 150 is configured to deform prior to breaking. For example, between point 606 and point 608, as the force increases, coupling element 150 may deform. At point 608, coupling element 150 may break causing coupler assembly 100 to be replaced to allow for the flow of fluid from fluid source 500 to patient end 600.

In some embodiments, the first predetermined threshold force is approximately 4 pounds (lbs). The first predetermined threshold force may be from approximately 1 lb to approximately 8 lbs, approximately 3 lbs to approximately 7 lbs, approximately 4 lbs to approximately 6 lbs, or greater than 8 lbs. In some embodiments, the second predetermined threshold force is approximately 6 lbs or greater. The second predetermined threshold force may be from approximately 6 lb to approximately 50 lbs, approximately 10 lbs to approximately 40 lbs, approximately 15 lbs to approximately 25 lbs, or greater than 6 lbs. In some embodiments, the first predetermined threshold force is less than the second predetermined threshold force. For example, the second predetermined threshold force may be 6 lbs and the first predetermined threshold may be 1 lb to 5 lbs.

The first predetermined force and the second predetermined force may vary based on the materials and/or dimensions used for first connector 102, second connector 102′, and/or coupling element 150. For example, the greater the strength of coupling element 150, the greater the second predetermined threshold needs to be to cause breakage of coupling element 150. For example, coupling element 150 being made out of a strong material may result in the second predetermined threshold being higher than coupling element 150 being made of a weaker, flexible material. Further, a greater length of coupling element 150 may result in an increase of second predetermined threshold force compared to coupling element 150 being shorter in length.

In practice, a patient may have a needle/catheter inserted into their skin and the needle/catheter may be coupled to first connector 102 or second connector 102′. The patient may walk away from an infusion pump or accidental pull on a fluid line coupled to first connector 102 or second connector 102′ and the force exceeds 4 lbs but is less than 6 lbs, first connector 102 or second connector 102′ may transition from the first configuration to the second configuration effectively closing the fluid pathway within first connector 102 or second connector 102′, as described herein. As the patient walks further away or tugs and pulls on the fluid line to first connector 102 or second connector 102′ and the force exceeds 6 lbs, first connector 102 or second connector 102′ may transition from the second configuration to the third configuration resulting in coupling element 150 breaking and coupler assembly 100 needing to be replaced, as described herein.

In some embodiments, upon breaking of coupling element 150, a user or medical professional removes first connector 102 and second connector 102′ and inserts a new coupler assembly 100 in the fluid line. In some embodiments, upon breaking of coupling element 150, a user or medical professional causes inner piece 107 and/or 107′ to retract into outer piece 105 and/or 105′, respectively, to cause first connector 102 and/or second connector 102′ to transition from the third configuration to the first configuration. The user or medical professional may replace coupling element 150 and couple first connector 102 to second connector 102′ with the replaced coupling element 150. The user or medical professional may further sterilize first connector 102 and second connector 102′.

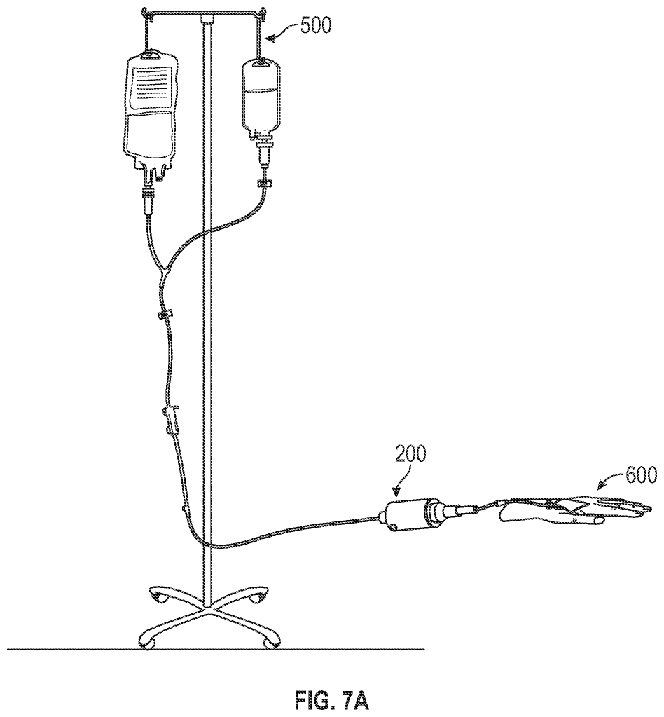

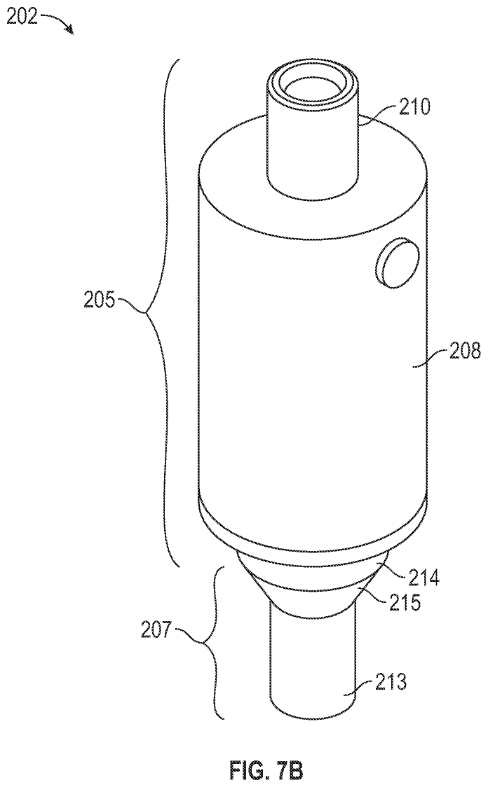

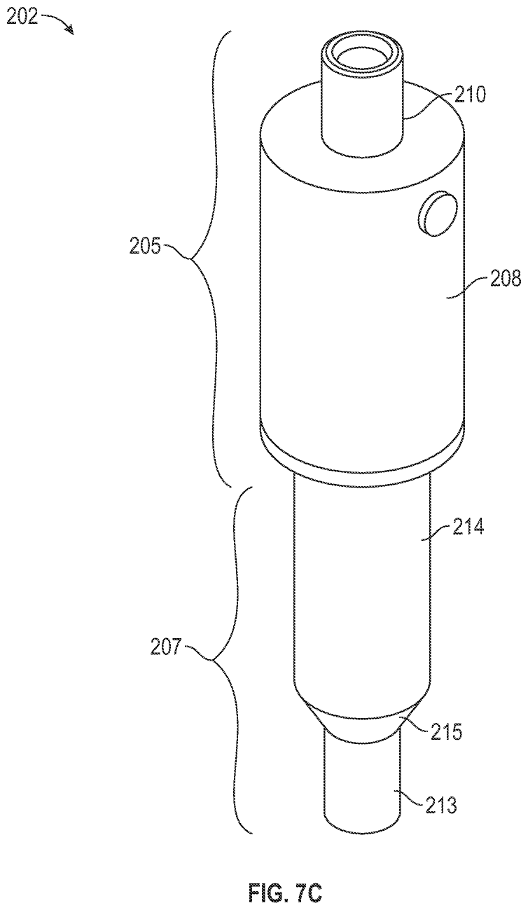

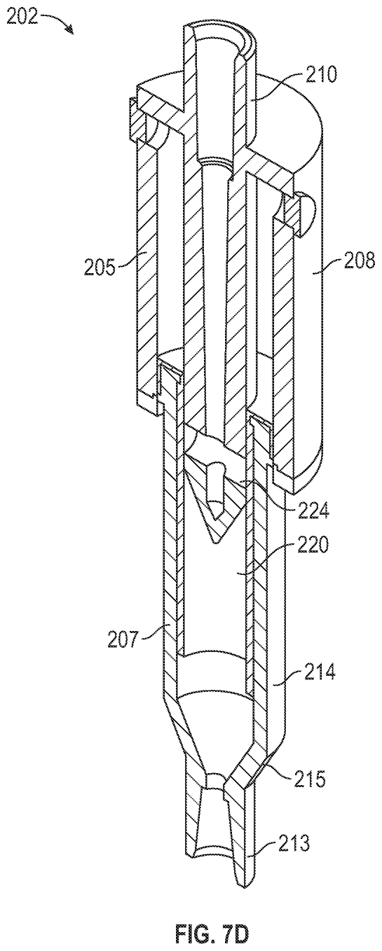

FIG. 7A is a system diagram showing a coupler assembly of a second embodiment in use, in accordance with various aspects of the present disclosure. FIG. 7B is a cross-sectional view of the connector of FIG. 7A with the connector in the first configuration, in accordance with various aspects of the present disclosure. FIG. 7C is a perspective front view of the connector of FIG. 7A with the connector in the second configuration, in accordance with various aspects of the present disclosure. FIG. 7D is a cross-sectional view of the connector of FIG. 7A with the connector in a third configuration, in accordance with various aspects of the present disclosure.

Referring to FIGS. 7A-7D, there is shown a second exemplary embodiment. Coupler assembly 200 may be similar to coupler assembly 100 as discussed herein but includes only first connector 202. First connector 202 may be similar to first connector 102 and second connector 102′, as discussed herein, but does not include locking tabs. Coupler assembly 200 may not include second connector 102′ or coupling element 150. For example, tubing portion 204 may be coupled to fluid source 500 via a first portion of tubing and coupling portion 213 is coupled to patient end 600 via a second portion of tubing. In some embodiments, coupler assembly 200 not include second connector 102′ or coupling element 150 results in coupler assembly 200 not breaking in response to a pullout force exceeding a second predetermined threshold force.

In some embodiments, first connector 202 does not include locking tabs 119 that are present in first connector 102. First connector 202 not including locking tabs 119 allows inner piece 207 to retract into outer piece 205 when inner piece 207 is in the fully extended position (FIG. 7C). In some embodiments, first connector 202 is configured to transition to the fully extended position, thereby blocking occlusion hole 224 with seal 220 and inner piece 207 in response to a pullout force exceeding the second predetermined threshold. When the pullout force reaches or exceeds the second predetermined threshold, there is no coupling element 150 to break, thus coupler assembly 200 does not break. In some embodiments, coupler assembly 200 is coupled to a device (e.g., pump or other medical device) such that occlusion of occlusion hole 224 and blocking of the fluid pathway within first connector 202 results in device generating an alarm. The generation of alarm is configured to alert a medical professional to reduce or eliminate the pullout force caused on first connector 202.

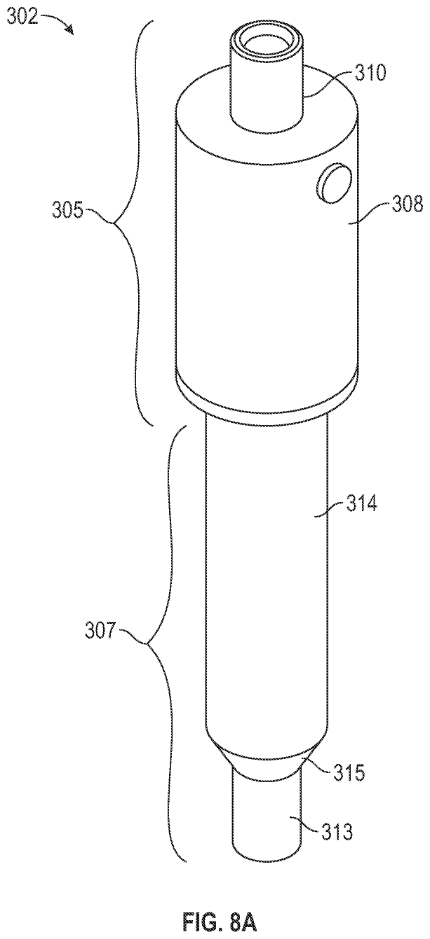

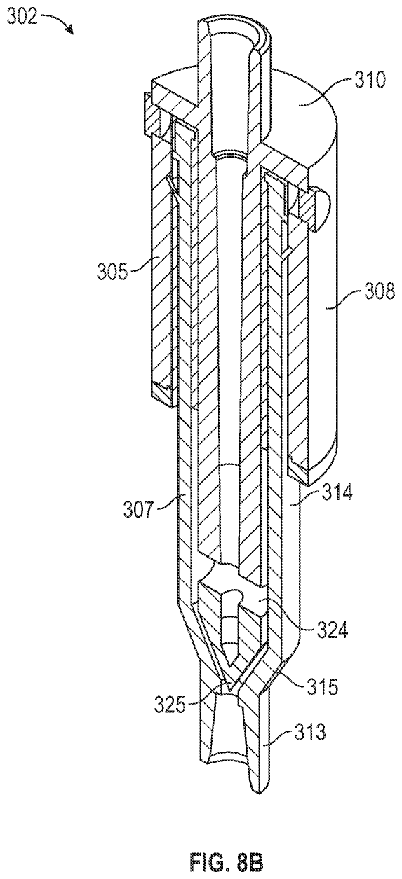

FIG. 8A is a cross-sectional view of a third embodiment of a connector of the coupler assembly of FIG. 1 with the connector in the first configuration, in accordance with various aspects of the present disclosure. FIG. 8B is a cross-sectional view of a third embodiment of a connector of the coupler assembly of FIG. 1 with the connector in the second configuration, in accordance with various aspects of the present disclosure.

Referring to FIGS. 8A-8B, there is shown a third exemplary embodiment. First connector 302 may be similar to first connector 102 and second connector 102′, as discussed herein, but extending portion 314 is longer than extending portion 114. In some embodiments, extending portion 314 being longer than extending portion 114 results in inner piece 307 being longer than inner piece 107. Further, extending portion 314 being longer than extending portion 114 results in distal end 325 and occlusion hole 324 being further away form first end 301 compared to distal end 125 and occlusion hole 124. In some embodiments, extending portion 314 being longer than extending portion allows for a larger buffer before occlusion or blockage of occlusion hole 324.

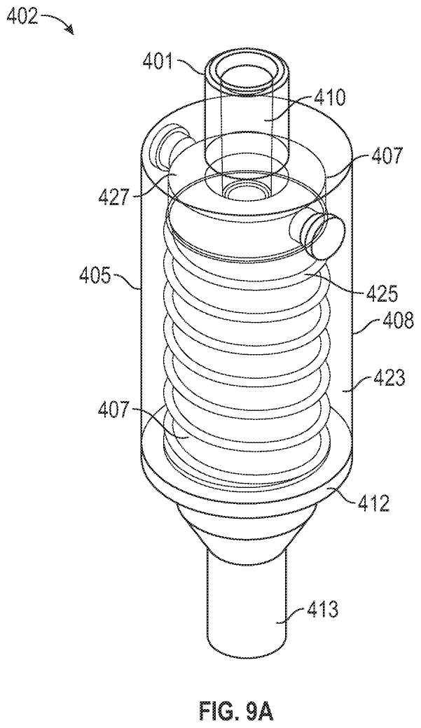

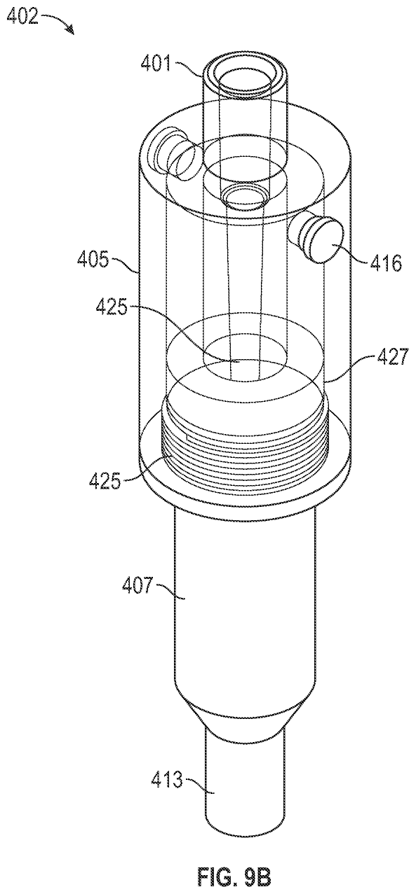

FIG. 9A is a cross-sectional view of a fourth embodiment of a connector of the coupler assembly of FIG. 1 with the connector in the first configuration, in accordance with various aspects of the present disclosure. FIG. 9B is a cross-sectional view of a fourth embodiment of a connector of the coupler assembly of FIG. 1 with the connector in the second configuration, in accordance with various aspects of the present disclosure.

Referring to FIGS. 9A-9B, there is shown a fourth exemplary embodiment. First connector 402 may be similar to first connector 102 and second connector 102′ as discussed herein, but first connector 402 may include biasing element 425. Biasing element 425 may be disposed within interior space 423 of outer piece 405. In some embodiments, biasing element 425 is configured to bias inner piece 407 in the initial position. For example, compared to first connector 102 and second connector 102′, which use a vacuum to bias inner piece 107 in the initial position, first connector 402 uses biasing element 425 to bias inner piece 407 in the initial position.

Biasing element 425 may be a spring configured to compress in response to a pullout force. Biasing element 425 may have a compressed state and an expanded state. Biasing element 425 may naturally be in the expanded state and may compress in response to force (e.g., pullout force). For example, a pullout force applied first connector 402 may result in inner piece 407 transition from the initial position to the partially extended or fully extending position. As inner piece 407 transitions to the partially extended or fully extended position, biasing element 425 may transition from the expanded state to the compressed state. In some embodiments, biasing element 425 is disposed between top edge 427 of seal 420 and locking ring 412 such that as inner piece 407 and seal 420 axially move away from first end 401 relative to outer piece 405, top edge 427 compresses biasing element 425. Upon reduction or elimination of the pullout force, biasing element 425 may return to the expanded state causing inner piece 407 to retract back into outer piece 405. In some embodiments, first connector 402 includes vents 416 instead of plugs 116 of first connector 102. For example, vents 416 may allow for air to travel into and out of outer piece 405 due to biasing element 425 compressing and expanding.

The disclosures described herein include at least the following clauses:

Clause 1: A coupler comprising a first connector including a first outer piece having a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position, the first channel including a first occlusion hole, and the first channel and the first occlusion hole forming a first fluid pathway through the first connector when the first inner piece is in the retracted position, wherein in the extended position the first occlusion hole is blocked by the first seal resulting in the first fluid pathway being blocked, the first inner piece transitioning to the partially extended position in response to a pullout force exceeding a first predetermined threshold.

Clause 2: The coupler of clause 1 further comprising a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position, the second channel including a second occlusion hole, and the second channel and the second occlusion hole forming a second fluid pathway through the second connector when the second inner piece is in the retracted position, wherein in the partially extended position the second occlusion hole is blocked by the second seal resulting in the second fluid pathway being blocked, the second inner piece transitioning to the partially extended position in response to the pullout force exceeding the first predetermined threshold.

Clause 3: The coupler of clause 2 further comprising a coupling element coupling the first connector to the second connector, the coupling element configured to cause the first fluid pathway to be in fluid communication with the second fluid pathway when the first connector is coupled to the second connector.

Clause 4: The coupler of clause 3, wherein the coupling element is configured to break into two or more portions in response to the pullout force exceeding a second predetermined threshold.

Clause 5: The coupler of clause 4, wherein the second predetermined threshold is greater than the first predetermined threshold.

Clause 6: The coupler of clause 3, wherein a central axis extends through the first connector, the coupling element, and the second connector when the first connector is coupled to the second connector via the coupling element.

Clause 7: The coupler of clause 3, wherein the first connector is configured to remain coupled to the coupling element and the second connector when the pullout force does not exceed the first predetermined threshold force.

Clause 8: The coupler of clause 3, wherein the coupler has a first configuration and in the first configuration the first connector is coupled to the second connector via the coupling element such that the first fluid pathway is in fluid communication with the second fluid pathway.

Clause 9: The coupler of clause 3, wherein the coupler has a second configuration and in the second configuration the first connector is coupled to the second connector and the first inner piece is in the partially extended position and the second inner piece is in the partially extended position such that the first fluid pathway is blocked and the second fluid pathway is blocked.

Clause 10: The coupler of clause 3, wherein the coupler has a third configuration and in the third configuration the coupling element is broken such that the first connector is decoupled from the second connector.

Clause 11: The coupler of clause 1, wherein the first inner piece includes a first coupling portion and the first inner piece is configured to transition from the partially extended position to a fully extended position, the fully extended position being where the first coupling portion is disposed further from the first occlusion hole compared to when the first inner piece is in the partially extended position.

Clause 12: The coupler of clause 11, wherein the first inner piece includes one or more locking tabs, the one or more locking tabs configured to prevent retraction of the first inner piece when the first inner piece is in the fully extended position.

Clause 13: The coupler of clause 11, wherein the first inner piece transitions from the partially extended position to the fully extended position in response the pullout force exceeding a second predetermined threshold, the second predetermined threshold being greater than the first predetermined threshold.

Clause 14: The coupler of clause 1, wherein the first outer piece includes a first interior space and the first inner piece is biased to be in the retracted into the first interior space due to a vacuum generated in the first interior space.

Clause 15: The coupler of clause 1, wherein the first outer piece includes a first tubing portion configured to couple to a portion of tubing and the first inner piece includes a first coupling portion configured to couple to a coupling element, the first tubing portion being in fluid communication the first coupling portion through the first channel and the first occlusion hole when the first inner piece is in the retracted position.

Clause 16: The coupler of clause 1, wherein a length of the first inner piece is greater than a length of the first outer piece.

Clause 17: The coupler of clause 1, wherein a maximum diameter of the first outer piece is greater than a maximum diameter of the first inner piece.

Clause 18: The coupler of clause 1 further comprising a locking ring secured to the first outer piece, the locking ring configured to prevent the first inner piece from withdrawing from the first outer piece.

Clause 19: The coupler of clause 1 further comprising a biasing element disposed within an interior space of the first outer piece and having a compressed state and an expanded state, the biasing element configured to bias the first inner piece to be in the retracted position, wherein the biasing element transitions from the expanded state to the compressed state in response to the pullout force exceeding the first predetermined threshold.

Clause 20: The coupler of clause 1, wherein the pullout force is a force applied to the first connector along a central axis of the first connector and the central axis extends at least along a length of the first connector.

Clause 21: A coupler comprising a first connector including a first outer piece having a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position, the first channel including a first occlusion hole, and the first channel and the first occlusion hole forming a first fluid pathway through the first connector when the first inner piece is in the retracted position, wherein in the partially extended position the first occlusion hole is blocked by the first seal resulting in the first fluid pathway being blocked, the first inner piece transitioning to the partially extended position in response to a pullout force exceeding a first predetermined threshold, a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position, the second channel including a second occlusion hole, and the second channel and the second occlusion hole forming a second fluid pathway through the second connector when the second inner piece is in the retracted position, wherein in the partially extended position the second occlusion hole is blocked by the second seal resulting in the second fluid pathway being blocked, the second inner piece transitioning to the partially extended position in response to the pullout force exceeding the first predetermined threshold, and a coupling element coupling the first connector to the second connector, the coupling element configured to cause the first fluid pathway to be in fluid communication with the second fluid pathway when the first connector is coupled to the second connector.

Clause 22: A coupler comprising a first connector including a first outer piece having a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position to a fully extended position, the first channel including a first occlusion hole substantially perpendicular to the first channel, and the first channel and the first occlusion hole forming a first fluid pathway through the first connector when the first inner piece is in the retracted position, wherein in the partially extended position the first occlusion hole is blocked by the first seal resulting in the first fluid pathway being blocked, the first inner piece transitioning to the partially extended position in response to a pullout force exceeding a first predetermined threshold, wherein the first inner piece transitions from the partially extended position to the fully extended position in response the pullout force exceeding a second predetermined threshold, a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position to a fully extended position, the second channel including a second occlusion hole substantially perpendicular to the second channel, and the second channel and the second occlusion hole forming a second fluid pathway through the second connector when the second inner piece is in the retracted position, wherein in the partially extended position the second occlusion hole is blocked by the second seal resulting in the second fluid pathway being blocked, the second inner piece transitioning to the partially extended position in response to the pullout force exceeding the first predetermined threshold, wherein the first inner piece transitions from the partially extended position to the fully extended position in response the pullout force exceeding a second predetermined threshold, and a coupling element coupling the first connector to the second connector, the coupling element configured to cause the first fluid pathway to be in fluid communication with the second fluid pathway when the first connector is coupled to the second connector, wherein the coupling element is configured to break into two or more portions in response to the pullout force exceeding the second predetermined threshold, the second predetermined threshold being greater than the first predetermined threshold, wherein the pullout force is a force applied to the first connector along a central axis of the first connector and the central axis extends at least along a length of the first connector, the second connector, and the coupling element.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A coupler comprising:
a first connector including a first outer piece having a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position, the first channel including a first occlusion hole, and the first channel and the first occlusion hole forming a first fluid pathway through the first connector when the first inner piece is in the retracted position,
wherein in the extended position the first occlusion hole is blocked by the first seal resulting in the first fluid pathway being blocked, the first inner piece transitioning to the partially extended position in response to a pullout force exceeding a first predetermined threshold.

2. The coupler of claim 1 further comprising:
a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position, the second channel including a second occlusion hole, and the second channel and the second occlusion hole forming a second fluid pathway through the second connector when the second inner piece is in the retracted position,
wherein in the partially extended position the second occlusion hole is blocked by the second seal resulting in the second fluid pathway being blocked, the second inner piece transitioning to the partially extended position in response to the pullout force exceeding the first predetermined threshold.

3. The coupler of claim 2 further comprising:
a coupling element coupling the first connector to the second connector, the coupling element configured to cause the first fluid pathway to be in fluid communication with the second fluid pathway when the first connector is coupled to the second connector.

4. The coupler of claim 3, wherein the coupling element is configured to break into two or more portions in response to the pullout force exceeding a second predetermined threshold.

5. The coupler of claim 4, wherein the second predetermined threshold is greater than the first predetermined threshold.

6. The coupler of claim 3, wherein a central axis extends through the first connector, the coupling element, and the second connector when the first connector is coupled to the second connector via the coupling element.

7. The coupler of claim 3, wherein the first connector is configured to remain coupled to the coupling element and the second connector when the pullout force does not exceed the first predetermined threshold force.

8. The coupler of claim 3, wherein the coupler has a first configuration and in the first configuration the first connector is coupled to the second connector via the coupling element such that the first fluid pathway is in fluid communication with the second fluid pathway.

9. The coupler of claim 3, wherein the coupler has a second configuration and in the second configuration the first connector is coupled to the second connector and the first inner piece is in the partially extended position and the second inner piece is in the partially extended position such that the first fluid pathway is blocked and the second fluid pathway is blocked.

10. The coupler of claim 3, wherein the coupler has a third configuration and in the third configuration the coupling element is broken such that the first connector is decoupled from the second connector.

11. The coupler of claim 1, wherein the first inner piece includes a first coupling portion and the first inner piece is configured to transition from the partially extended position to a fully extended position, the fully extended position being where the first coupling portion is disposed further from the first occlusion hole compared to when the first inner piece is in the partially extended position.

12. The coupler of claim 11, wherein the first inner piece includes one or more locking tabs, the one or more locking tabs configured to prevent retraction of the first inner piece when the first inner piece is in the fully extended position.

13. The coupler of claim 11, wherein the first inner piece transitions from the partially extended position to the fully extended position in response the pullout force exceeding a second predetermined threshold, the second predetermined threshold being greater than the first predetermined threshold.

14. The coupler of claim 1, wherein the first outer piece includes a first interior space and the first inner piece is biased to be in the retracted into the first interior space due to a vacuum generated in the first interior space.

15. The coupler of claim 1, wherein the first outer piece includes a first tubing portion configured to couple to a portion of tubing and the first inner piece includes a first coupling portion configured to couple to a coupling element, the first tubing portion being in fluid communication the first coupling portion through the first channel and the first occlusion hole when the first inner piece is in the retracted position.

16. The coupler of claim 1 further comprising:
a locking ring secured to the first outer piece, the locking ring configured to prevent the first inner piece from withdrawing from the first outer piece.

17. The coupler of claim 1 further comprising:
a biasing element disposed within an interior space of the first outer piece and having a compressed state and an expanded state, the biasing element configured to bias the first inner piece to be in the retracted position, wherein the biasing element transitions from the expanded state to the compressed state in response to the pullout force exceeding the first predetermined threshold.

18. The coupler of claim 1, wherein the pullout force is a force applied to the first connector along a central axis of the first connector and the central axis extends at least along a length of the first connector.

19. A coupler comprising:
a first connector including a first outer piece having a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position, the first channel including a first occlusion hole, and the first channel and the first occlusion hole forming a first fluid pathway through the first connector when the first inner piece is in the retracted position, wherein in the partially extended position the first occlusion hole is blocked by the first seal resulting in the first fluid pathway being blocked, the first inner piece transitioning to the partially extended position in response to a pullout force exceeding a first predetermined threshold;
a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position, the second channel including a second occlusion hole, and the second channel and the second occlusion hole forming a second fluid pathway through the second connector when the second inner piece is in the retracted position, wherein in the partially extended position the second occlusion hole is blocked by the second seal resulting in the second fluid pathway being blocked, the second inner piece transitioning to the partially extended position in response to the pullout force exceeding the first predetermined threshold; and
a coupling element coupling the first connector to the second connector, the coupling element configured to cause the first fluid pathway to be in fluid communication with the second fluid pathway when the first connector is coupled to the second connector.

20. A coupler comprising:
a first connector including a first outer piece having a first channel extending longitudinally within the first outer piece, a first inner piece disposed at least partially within the first outer piece such that the first channel is disposed within the first inner piece, and a first seal disposed within the first inner piece such that the first seal is at least partially disposed between the first channel and the first inner piece, the first inner piece configured to axially move relative to the first outer piece to transition from a retracted position to a partially extended position to a fully extended position, the first channel including a first occlusion hole substantially perpendicular to the first channel, and the first channel and the first occlusion hole forming a first fluid pathway through the first connector when the first inner piece is in the retracted position, wherein in the partially extended position the first occlusion hole is blocked by the first seal resulting in the first fluid pathway being blocked, the first inner piece transitioning to the partially extended position in response to a pullout force exceeding a first predetermined threshold, wherein the first inner piece transitions from the partially extended position to the fully extended position in response the pullout force exceeding a second predetermined threshold;
a second connector including a second outer piece having a second channel extending longitudinally within the second outer piece, a second inner piece disposed at least partially within the second outer piece such that the second channel is disposed within the second inner piece, and a second seal disposed within the second inner piece such that the second seal is at least partially disposed between the second channel and the second inner piece, the second inner piece configured to axially move relative to the second outer piece to transition from a retracted position to a partially extended position to a fully extended position, the second channel including a second occlusion hole substantially perpendicular to the second channel, and the second channel and the second occlusion hole forming a second fluid pathway through the second connector when the second inner piece is in the retracted position, wherein in the partially extended position the second occlusion hole is blocked by the second seal resulting in the second fluid pathway being blocked, the second inner piece transitioning to the partially extended position in response to the pullout force exceeding the first predetermined threshold, wherein the first inner piece transitions from the partially extended position to the fully extended position in response the pullout force exceeding a second predetermined threshold; and
a coupling element coupling the first connector to the second connector, the coupling element configured to cause the first fluid pathway to be in fluid communication with the second fluid pathway when the first connector is coupled to the second connector,
wherein the coupling element is configured to break into two or more portions in response to the pullout force exceeding the second predetermined threshold, the second predetermined threshold being greater than the first predetermined threshold,
wherein the pullout force is a force applied to the first connector along a central axis of the first connector and the central axis extends at least along a length of the first connector, the second connector, and the coupling element.
